(19) United States
(12) Patent Application Publication
(10) Pub. No.: US 2023/0381401 A1
(43) Pub. Date: Nov. 30, 2023

(54) NEGATIVE-PRESSURE THERAPY DRESSING WITH VIEWING WINDOW

(71) Applicant: KCI Manufacturing Unlimited Company, Westmeath (IE)

(72) Inventor: Christopher Brian LOCKE, Bournemouth (GB)

(21) Appl. No.: 18/020,565

(22) PCT Filed: Jul. 26, 2021
(86) PCT No.: PCT/IB2021/056741

§ 371 Date: Feb. 9, 2023

Related U.S. Application Data
(60) Provisional application No. 63/065,223, filed on Aug. 13, 2020.

(51) Int. Cl. A61M 1/00; A61F 13/02

(52) U.S. Cl. CPC A61M 1/915; A61F 13/0216; A61F 13/0289; A61M 1/918; A61F 2013/00846

(57) ABSTRACT

Disclosed embodiments may relate to dressings configured to provide negative-pressure therapy to a tissue site, such as an incision, while simultaneously allowing viewing of the tissue site during negative-pressure therapy. For example, the dressing may comprise a viewing window extending through the manifold. In some embodiments, the viewing window may be configured to provide lateral strain and/or apposition forces on the tissue site during therapy. In some embodiments, the manifold may comprise two horizontally-spaced strips of foam with a gap therebetween, and a transparent separator film may span the gap. In some embodiments, the separator film may comprise a plurality of perforations and/or textured features. In some embodiments, a transparent cover may be disposed over the manifolding strips. Additionally disclosed are other apparatus, dressings, systems, and methods.

### CROSS-REFERENCE TO RELATED APPLICATIONS

This application claims the benefit of priority to U.S. Provisional Application No. 60/065,223 filed on Aug. 13, 2020, which is incorporated herein by reference in its entirety.

### TECHNICAL FIELD

This disclosure relates generally to tissue treatment systems and more particularly, but without limitation, to dressings, systems, and methods relating to negative-pressure therapy.

### BACKGROUND

Clinical studies and practice have shown that reducing pressure in proximity to a tissue site can augment and accelerate growth of new tissue at the tissue site. The applications of this phenomenon are numerous, but it has proven particularly advantageous for treating wounds. Regardless of the etiology of a wound, whether trauma, surgery, or another cause, proper care of the wound is important to the outcome. Treatment of wounds or other tissue with reduced pressure may be commonly referred to as “negative-pressure therapy,” but is also known by other names, including “negative-pressure wound therapy,” “reduced-pressure therapy,” “vacuum therapy,” “vacuum-assisted closure,” and “topical negative-pressure,” for example. Reduced-pressure therapy may provide a number of benefits, including migration of epithelial and subcutaneous tissues, improved blood flow, and micro-deformation of tissue at a wound site. Together, these benefits can increase development of granulation tissue and reduce healing times.

While the clinical benefits of negative-pressure therapy are widely known, improvements to therapy systems, components, and processes may benefit healthcare providers and patients.

### BRIEF SUMMARY

New and useful systems, apparatuses, and methods for managing tissue sites in a negative-pressure therapy environment are set forth in the appended claims. The following description provides non-limiting, illustrative example embodiments to enable a person skilled in the art to make and use the claimed subject matter.

Disclosed embodiments may relate to dressings configured to provide negative-pressure therapy to a tissue site, such as an incision, while simultaneously allowing viewing of the tissue site during negative-pressure therapy. For example, the dressing may comprise a viewing window extending through the manifold. In some embodiments, the viewing window may be configured to provide lateral strain and/or apposition forces on the tissue site during therapy. In some embodiments, the viewing widow may be configured to provide permanent and/or unobstructed visualization of the incision, wound, and/or tissue site, even during therapy. In some embodiments, the manifold may comprise two horizontally-spaced strips of foam with a gap therebetween, and a transparent separator film may span the gap. In some embodiments, the separator film may comprise a plurality of perforations and/or textured features, such as one or more longitudinal ridges which may be configured to offer some lateral collapse when subjected to negative pressure. In some embodiments, a transparent cover may be disposed over the manifolding strips.

In some example embodiments, a dressing for applying negative-pressure therapy at a tissue site may comprise: a manifold comprising two horizontally-spaced strips of manifolding material, such as open-cell foam; a gap between the two strips of manifolding material; and a separator film spanning the gap and bonded to the two strips of manifolding material; wherein the separator film may be substantially transparent. In some embodiments, the separator film may comprise a plurality of perforations, for example substantially spanning the separator film. In some embodiments, the separator film may be configured with textured features, such as one or more thermoformed longitudinal ridges. Some embodiments may further comprise a protective layer adjacent to a first surface of the two strips of manifolding material. The protective layer may be configured to prevent in-growth into the strips of manifolding material, in some embodiments, while allowing fluid communication between the strips of manifolding material and the tissue site. In some embodiments, the two strips of manifolding material may be joined at one or both ends, and/or the gap may not extend the full length of the two strips of manifolding material. Some embodiments may further comprise a cover configured to be disposed over the two strips of manifolding material and to substantially prevent fluid flow therethrough. Typically, the cover may be substantially transparent, and the vertically aligned portion of the transparent cover and the transparent separator film may form the viewing window through the dressing.

In some example embodiments, a system for providing negative-pressure therapy to a tissue site may comprise: a negative-pressure source; and a dressing, with the negative-pressure source fluidly coupled to the dressing and configured to provide negative-pressure therapy to a tissue site through the dressing. In some embodiments, the dressing may comprise: a manifold having two horizontally-spaced strips of manifolding material; a gap between the two strips of manifolding material; a separator film spanning the gap and bonded to the two strips of manifolding material; and a cover configured to be disposed over the manifold and to substantially prevent fluid flow therethrough. In some embodiments, the separator film and the cover may each be substantially transparent; and/or the negative-pressure source may be fluidly coupled to the manifold through the cover. In some embodiments, the separator film may comprise a plurality of perforations substantially coextensive with the separator film and/or may be configured with textured features, which may comprise one or more thermoformed longitudinal ridges. Some embodiments may further comprise a protective layer adjacent to the first surface of the two strips of manifolding material, which may be configured to prevent in-growth into the strips of manifolding material while allowing fluid communication between the strips of manifolding material and the tissue site.

In some example embodiments, a method, for providing negative-pressure therapy to an incision, may comprise: positioning a dressing with a viewing window over the incision; and applying negative pressure through the dressing to the incision, wherein the negative pressure collapses the dressing vertically and laterally in the horizontal direction and induces lateral strain and/or appositional forces on the incision. Some embodiments may further comprise viewing the incision through the viewing window during application of negative pressure.

In some example embodiments, a method of manufacturing a negative-pressure dressing may comprise: providing two manifolding strips; disposing the two manifolding strips laterally side-by-side with a gap therebetween; providing a separator film; disposing the separator film to span the gap; and bonding the separator film to each of the manifolding strips, for example to a first surface of the two strips. In some embodiments, providing the separator film may comprise: providing a substantially transparent film; forming perforations in the film; and forming one or more textured features in the film configured to provide lateral strain and/or appositional forces when under negative pressure.

Objectives, advantages, and a preferred mode of making and using the claimed subject matter may be understood best by reference to the accompanying drawings in conjunction with the following detailed description of illustrative example embodiments.

### BRIEF DESCRIPTION OF THE DRAWINGS

FIG. 1 is a block diagram of an example embodiment of a therapy system that can provide negative-pressure therapy in accordance with this specification;

FIG. 2 is a graph illustrating example pressure control modes that may be associated with some example embodiments of the therapy system of FIG. 1;

FIG. 3 is a graph illustrating another example pressure control mode suitable for some example embodiments of the therapy system of FIG. 1;

FIG. 4 is an exploded, isometric view of an example embodiment of a dressing that may be associated with an example embodiment of the therapy system of FIG. 1;

FIG. 5 is a schematic cross-section view illustrating an exemplary system having an exemplary dressing in place on an exemplary tissue site, illustrating additional details that may be associated with some embodiments;

FIG. 6 is an isometric view of an example tissue interface for a dressing for use with the therapy system of FIG. 1, illustrating additional details that may be associated with some embodiments;

FIG. 7 is a schematic cross-section view of an example dressing having the tissue interface of FIG. 6, illustrating additional details that may be associated with some embodiments;

FIG. 8 is a schematic cross-section view of the dressing of FIG. 7 during negative-pressure therapy on a tissue site, illustrating additional details that may be associated with some embodiments;

FIG. 9 is an isometric view of another example dressing similar to that of FIG. 7 for use with the therapy system of FIG. 1, illustrating additional details that may be associated with some embodiments;

FIG. 10 is a bottom plan view of another example tissue interface, illustrating additional details that may be associated with some embodiments; and

FIG. 11 is a partial isometric view of an example separator film that may be associated with the tissue interface of FIG. 6 or FIG. 10, illustrating additional details that may be associated with some embodiments.

### DESCRIPTION OF EXAMPLE EMBODIMENTS

The following description of example embodiments provides information that enables a person skilled in the art to make and use the subject matter set forth in the appended claims, but may omit certain details already well-known in the art. The following detailed description is, therefore, to be taken as illustrative and non-limiting.

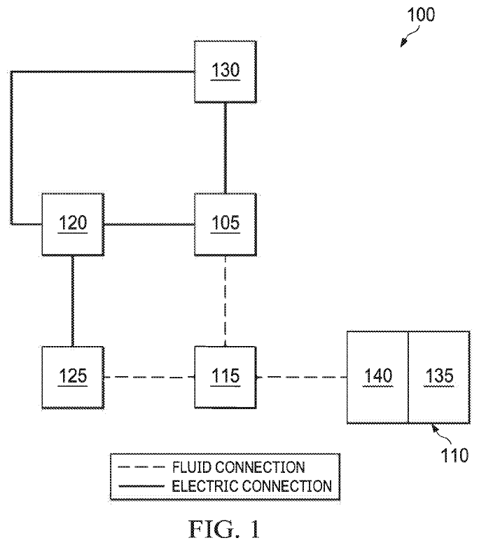

FIG. 1 is a block diagram of an example embodiment of a therapy system 100 that can provide negative-pressure therapy to a tissue site in accordance with this specification. The term “tissue site” in this context may refer to a wound, defect, or other treatment target located on or within tissue, including but not limited to, bone tissue, adipose tissue, muscle tissue, neural tissue, dermal tissue, vascular tissue, connective tissue, cartilage, tendons, or ligaments. A wound may include chronic, acute, traumatic, subacute, and dehisced wounds, partial-thickness burns, ulcers (such as diabetic, pressure, or venous insufficiency ulcers), flaps, grafts, and incisions, for example. The term “tissue site” may also refer to areas of any tissue that are not necessarily wounded or defective, but are instead areas in which it may be desirable to add or promote the growth of additional tissue. For example, negative pressure may be applied to a tissue site to grow additional tissue that may be harvested and transplanted.

The therapy system 100 may include a source or supply of reduced or negative pressure, such as a negative-pressure source 105, a dressing 110, a fluid container, such as a container 115, and a regulator or controller, such as a controller 120, for example. Additionally, the therapy system 100 may include sensors to measure operating parameters and provide feedback signals to the controller 120 indicative of the operating parameters. As illustrated in FIG. 1, for example, the therapy system 100 may include one or more sensors coupled to the controller 120, such as a first sensor 125 and a second sensor 130. As illustrated in the example of FIG. 1, the dressing 110 may include a tissue interface 135, a cover 140, or both in some embodiments. The dressing 110 may also be referred to as a dressing assembly in some examples, which may include additional or different features as described herein.

Some components of the therapy system 100 may be housed within or used in conjunction with other components, such as sensors, processing units, alarm indicators, memory, databases, software, display devices, or user interfaces that further facilitate therapy. For example, in some embodiments, the negative-pressure source 105 may be combined with the controller 120 and other components into a therapy unit.

In general, components of the therapy system 100 may be coupled directly or indirectly. For example, the negative-pressure source 105 may be directly coupled to the container 115, and may be indirectly coupled to the dressing 110 through the container 115. Coupling may include fluid, mechanical, thermal, electrical, or chemical coupling (such as a chemical bond), or some combination of coupling in some contexts. For example, the negative-pressure source 105 may be electrically coupled to the controller 120, and may be fluidly coupled to one or more distribution components to provide a fluid path to a tissue site. In some embodiments, components may also be coupled by virtue of physical proximity, being integral to a single structure, or being formed from the same piece of material.

A distribution component may be detachable, and may be disposable, reusable, or recyclable. The dressing 110 and the container 115 are illustrative of distribution components. A fluid conductor is another illustrative example of a distribution component. A “fluid conductor,” in this context, may include a tube, pipe, hose, conduit, or other structure with one or more lumina or open pathways adapted to convey a fluid between two ends. Typically, a tube is an elongated, cylindrical structure with some flexibility, but the geometry and rigidity may vary. Moreover, some fluid conductors may be molded into or otherwise integrally combined with other components. Distribution components may also include interfaces or fluid ports to facilitate coupling and de-coupling other components. In some embodiments, for example, a dressing interface may facilitate coupling a fluid conductor to the dressing 110. For example, such a dressing interface may be a SENSAT.R.A.C.™ Pad available from KCI of San Antonio, Texas.

A negative-pressure supply, such as the negative-pressure source 105, may be a reservoir of air at a reduced pressure, or may be a manual or electrically-powered device, such as a vacuum pump, a suction pump, a wall suction port available at many healthcare facilities, or a micro-pump, for example. “Negative pressure” or “reduced pressure” generally refers to a pressure less than a local ambient pressure, such as the ambient pressure in a local environment external to a sealed therapeutic environment. In many cases, the local ambient pressure may also be the atmospheric pressure at which a tissue site is located. Further, the pressure may be less than a hydrostatic pressure associated with tissue at the tissue site. Unless otherwise indicated, values of pressure stated herein are gauge pressures. References to increases in reduced pressure may refer to a decrease in absolute pressure, while decreases in reduced pressure may refer to an increase in absolute pressure. While the amount and nature of reduced pressure applied to a tissue site may vary according to therapeutic requirements, the pressure is generally a low vacuum, also commonly referred to as a rough vacuum, between −5 mm Hg (−667 Pa) and −500 mm Hg (−66.7 kPa). Common therapeutic ranges are between −50 mm Hg (−6.7 kPa) and −300 mm Hg (−39.9 kPa).

The container 115 is representative of a container, canister, pouch, or other storage component, which can be used to manage exudates and other fluids withdrawn from a tissue site. In many environments, a rigid container may be preferred or required for collecting, storing, and disposing of fluids. In other environments, fluids may be properly disposed of without rigid container storage, and a re-usable container could reduce waste and costs associated with negative-pressure therapy.

A controller, such as the controller 120, may be a microprocessor or computer programmed to operate one or more components of the therapy system 100, such as the negative-pressure source 105. In some embodiments, for example, the controller 120 may be a microcontroller, which may include an integrated circuit containing a processor core and a memory programmed to directly or indirectly control one or more operating parameters of the therapy system 100. Operating parameters may include the power applied to the negative-pressure source 105, the pressure generated by the negative-pressure source 105, or the pressure distributed to the tissue interface 135, for example. The controller 120 may also be configured to receive one or more input signals, such as a feedback signal, and programmed to modify one or more operating parameters based on the input signals.

Sensors, such as the first sensor 125 and the second sensor 130, may be any apparatus operable to detect or measure a physical phenomenon or property, and generally provide a signal indicative of the phenomenon or property that is detected or measured. For example, the first sensor 125 and the second sensor 130 may be configured to measure one or more operating parameters of the therapy system 100. In some embodiments, the first sensor 125 may be a transducer configured to measure pressure in a pneumatic pathway and convert the measurement to a signal indicative of the pressure measured. In some embodiments, for example, the first sensor 125 may be a piezoresistive strain gauge. The second sensor 130 may optionally measure operating parameters of the negative-pressure source 105, such as the voltage or current, in some embodiments. Signals from the first sensor 125 and the second sensor 130 may be suitable as an input signal to the controller 120, but some signal conditioning may be appropriate in some embodiments. For example, the signal may need to be filtered or amplified before it can be processed by the controller 120. Typically, the signal is an electrical signal, but may be represented in other forms, such as an optical signal.

The tissue interface 135 can be adapted to partially or fully contact a tissue site. The tissue interface 135 may take many forms, and may have many sizes, shapes, or thicknesses depending on a variety of factors, such as the type of treatment being implemented or the nature and size of a tissue site. For example, the size and shape of the tissue interface 135 may be adapted to the contours of deep and irregular shaped tissue sites. Moreover, any or all of the surfaces of the tissue interface 135 may have projections or an uneven, course, or jagged profile that can induce strains and stresses on a tissue site, which can promote granulation at the tissue site.

In some embodiments, the tissue interface 135 may be a manifold or may include a manifold and additional layers, components, or features, such as a tissue contact layer, depending on the desired treatment. A “manifold” in this context may include any substance or structure providing a plurality of pathways adapted to collect or distribute fluid relative to a tissue. For example, a manifold may be adapted to receive reduced pressure from a source and distribute reduced pressure through multiple apertures to or from a tissue site, which may have the effect of collecting fluid from a tissue site and drawing the fluid toward the source. In some embodiments, the fluid path may be reversed or a secondary fluid path may be provided to facilitate delivering or moving fluid relative to a tissue site.

In some illustrative embodiments, the pathways of a manifold may be interconnected to improve distribution or collection of fluids at a tissue site. In some illustrative embodiments, a manifold may be a porous foam material having interconnected cells or pores. For example, open-cell foam, porous tissue collections, and other porous material such as gauze or felted mat generally include pores, edges, and/or walls adapted to form interconnected fluid channels. Liquids, gels, and other foams may also include or be cured to include apertures and fluid pathways. In some embodiments, a manifold may additionally or alternatively include projections that form interconnected fluid pathways. For example, a manifold may be molded to provide surface projections that define interconnected fluid pathways.

The average pore size of foam may vary according to needs of a prescribed therapy. For example, in some embodiments, the tissue interface 135 may be foam having pore sizes in a range of 400-600 microns. The tensile strength of the tissue interface 135 may also vary according to needs of a prescribed therapy. For example, the tensile strength of foam may be increased for instillation of topical treatment solutions. In some examples, the tissue interface 135 may be reticulated polyurethane foam such as found in GRANUFOAM™ dressing or V.A.C. VERAFLO™ dressing, both available from KCI of San Antonio, Texas.

The tissue interface 135 may be either hydrophobic or hydrophilic. In an example in which the tissue interface 135 may be hydrophilic, the tissue interface 135 may also wick fluid away from a tissue site, while continuing to distribute negative pressure to the tissue site. The wicking properties of the tissue interface 135 may draw fluid away from a tissue site by capillary flow or other wicking mechanisms. An example of hydrophilic foam is a polyvinyl alcohol, open-cell foam such as V.A.C. WHITEFOAM™ dressing available from KCI of San Antonio, Texas. Other hydrophilic foams may include those made from polyether. Other foams that may exhibit hydrophilic characteristics include hydrophobic foams that have been treated or coated to provide hydrophilicity.

The tissue interface 135 may further promote granulation at a tissue site when pressure within the sealed therapeutic environment is reduced. For example, any or all of the surfaces of the tissue interface 135 may have an uneven, coarse, or jagged profile that can induce microstrain and stress at a tissue site if negative pressure is applied through the tissue interface 135.

In some embodiments, the tissue interface 135 may be constructed from bioresorbable materials. Suitable bioresorbable materials may include, without limitation, a polymeric blend of polylactic acid (PLA) and polyglycolic acid (PGA). The polymeric blend may also include without limitation polycarbonates, polyfumarates, and capralactones. The tissue interface 135 may further serve as a scaffold for new cell-growth, or a scaffold material may be used in conjunction with the tissue interface 135 to promote cell-growth. A scaffold is generally a substance or structure used to enhance or promote the growth of cells or formation of tissue, such as a three-dimensional porous structure that provides a template for cell growth. Illustrative examples of scaffold materials include calcium phosphate, collagen, PLA/PGA, coral hydroxy apatites, carbonates, or processed allograft materials.

Some embodiments of the tissue interface 135 may comprise layers, components, or features in addition to the manifold. For example, the tissue interface 135 of an absorptive dressing may comprise an absorbent layer, which may be characterized as exhibiting absorbency and/or as being adapted to absorb liquid (such as exudate) from the tissue site. In some embodiments, the absorbent layer may also be adapted to transfer negative pressure therethrough. In some embodiments, the absorbent layer may be configured to retain exudate and/or other fluids drawn from the tissue site during negative-pressure therapy, which may negate the necessity for separate fluid storage components such as an external fluid container. The absorbent layer may comprise any material capable of absorbing liquid (e.g. any absorbent material). In some embodiments, the absorbent layer may exhibit absorbency of at least 3 g saline/g, or at least 5 g saline/g, or from 8 to 20 g saline/g. In some embodiments, the absorbent layer may comprise superabsorbent material, such as superabsorbent polymer (SAP) particles or fibers. For example, some embodiments of the absorbent layer may comprise or consist essentially of one of the following: polyacrylate, sodium polyacrylate, polyacrylamide copolymer, ethylene-maleic anhydride copolymer, polyvinyl alcohol copolymer, cross-linked hydrophilic polymers, and combinations thereof. In some embodiments, the absorbent layer may be hydrophilic. In an example in which the absorbent layer is hydrophilic, the absorbent layer may also absorb or wick fluid away from one or more other components or layers of the dressing 110. In such an embodiment, the wicking properties of the absorbent layer may draw fluid away from one or more components or layers of the dressing 110 by capillary flow or other wicking mechanisms. An example of hydrophilic foam is a polyvinyl alcohol, open-cell foam. Other hydrophilic foams may include those made from polyether. Other foams that may exhibit hydrophilic characteristics include hydrophobic foams that have been treated or coated to provide hydrophilicity.

In some embodiments, the absorbent layer may have a bag-like structure for holding superabsorbent material. For example, the absorbent layer may be configured with superabsorbent material within a wicking pouch. In some embodiments, the pouch may comprise a first wicking layer and a second wicking layer. In some embodiments, the first wicking layer and the second wicking layer may be coupled around the pouch perimeter to form the enclosed pouch encapsulating (e.g. securely holding) the superabsorbent material to contain and prevent the superabsorbent material from migrating out of the pouch. For example, the first and second wicking layers may be coupled to each other using adhesive. The wicking layers may each comprise wicking material. The wicking material may be configured to be permeable to liquid (such as exudate), while retaining the superabsorbent material within the pouch. For example, the porosity of the wicking layers may be sufficiently small to prevent migration of the superabsorbent material through the wicking layers. The wicking layers may be configured to wick liquid along the superabsorbent material in a lateral direction normal to a thickness of the superabsorbent material within the pouch. Wicking of liquid laterally may enhance the distribution of liquid to the superabsorbent material, which may in turn speed absorption and/or allow for the superabsorbent material to maximize its absorbency. Examples of the wicking material may comprise or consist essentially of one of the following: Viscose, PET, Lidro™ non-woven material, a knitted polyester woven textile material, such as the one sold under the name InterDry® AG material from Coloplast A/S of Denmark, GORTEX® material, DuPont Softesse® material, etc., and combinations thereof. In some embodiments, the absorbent layer may serve as the manifold. For example, the absorbent layer may have manifolding properties, such that a separate manifold may not be necessary for negative-pressure therapy.

Some embodiments of the tissue interface 135 may comprise a protective layer (e.g. a tissue-contact layer). In some embodiments, the protective layer may act as a comfort layer, configured to improve comfort at the tissue site. In some embodiments, the protective layer may act as a fluid control layer, configured to minimize maceration, backflow of exudate out of the dressing to the tissue site, and/or tissue in-growth from the tissue site into the dressing 110. The protective layer may be configured to allow fluid transport from the tissue site into the dressing 110 and/or to manifold during negative-pressure therapy. In some embodiments, the protective layer may be configured as the tissue-contact surface for the dressing, so that in use it may be located adjacent to and/or direct contact with the tissue site. In some embodiments, the protective layer may be located between the tissue-contact surface and the manifold and/or the absorbent layer. In some embodiments, the protective layer may be located between the tissue site (when the dressing is in use) and the manifold and/or absorbent layer.

In some embodiments, the protective layer may comprise a porous fabric, a porous film, or a polymeric film (e.g. which may be liquid impermeable) with a plurality of fluid passages (e.g. slits, slots, or fluid valves). In some embodiments, the protective layer may comprise or consist essentially of a woven, elastic material or a polyester knit textile substrate. As a non-limiting example, an InterDry.™ textile material from Milliken Chemical of Spartanburg, South Carolina, may be used. The protective layer may also include anti-microbial substances, such as silver, in some embodiments.

In some embodiments, the protective layer may comprise or consist essentially of a liquid-impermeable, elastomeric material. For example, the protective layer may comprise or consist essentially of a polymer film. In some embodiments, for example, the protective layer may comprise or consist essentially of a hydrophobic polymer, such as a polyethylene film. The simple and inert structure of polyethylene can provide a surface that interacts little, if any, with biological tissues and fluids, providing a surface that may encourage the free flow of liquids and low adherence, which can be particularly advantageous for many applications. Other suitable polymeric films include polyurethanes, acrylics, polyolefin (such as cyclic olefin copolymers), polyacetates, polyamides, polyesters, copolyesters, PEBAX block copolymers, thermoplastic elastomers, thermoplastic vulcanizates, polyethers, polyvinyl alcohols, polypropylene, polymethylpentene, polycarbonate, styreneics, silicones, fluoropolymers, and acetates. A thickness between 20 microns and 100 microns may be suitable for many applications. In some embodiments, the protective layer may be hydrophobic. In some embodiments, the protective layer may be hydrophilic. In some embodiments, the protective layer may be suitable for coupling, such as welding, to other layers, such as the manifold.

Some embodiments of the protective layer may have one or more fluid passages, which can be distributed uniformly or randomly across the protective layer. The fluid passages may be bi-directional and pressure-responsive. For example, each of the fluid passages generally may comprise or consist essentially of an elastic passage that is normally unstrained to substantially reduce liquid flow, and can expand or open in response to a pressure gradient. In some embodiments, the fluid passage may comprise or consist essentially of perforations in the protective layer. Perforations may be formed by removing material from the protective layer. For example, perforations may be formed by cutting through the protective layer, which may also deform the edges of the perforations in some embodiments. In the absence of a pressure gradient across the perforations, the passages may be sufficiently small to form a seal or fluid restriction, which can substantially reduce or prevent liquid flow. Additionally or alternatively, one or more of the fluid passages may be an elastomeric valve that is normally closed when unstrained to substantially prevent liquid flow, and can open in response to a pressure gradient. A fenestration may be a suitable valve for some applications. Fenestrations may also be formed by removing material from the protective layer, but the amount of material removed and the resulting dimensions of the fenestrations may be up to an order of magnitude less than perforations, and may not deform the edges.

For example, some embodiments of the fluid passages may comprise or consist essentially of one or more slits, slots or combinations of slits and slots in the protective layer. In some examples, the fluid passages may comprise or consist of linear slots having a length less than 4 millimeters and a width less than 1 millimeter. The length may be at least 2 millimeters, and the width may be at least 0.4 millimeters in some embodiments. A length of about 3 millimeters and a width of about 0.8 millimeters may be particularly suitable for many applications, and a tolerance of about 0.1 millimeter may also be acceptable. Such dimensions and tolerances may be achieved with a laser cutter, for example. Slots of such configurations may function as imperfect valves that substantially reduce liquid flow in a normally closed or resting state. For example, such slots may form a flow restriction without being completely closed or sealed. The slots can expand or open wider in response to a pressure gradient to allow increased liquid flow.

In some embodiments, the cover 140 may provide a bacterial barrier and protection from physical trauma. The cover 140 may also be constructed from a material that can reduce evaporative losses and provide a fluid seal between two components or two environments, such as between a therapeutic environment and a local external environment. For example, the cover 140 may comprise or consist essentially of an elastomeric film or membrane that can provide a seal adequate to maintain a reduced pressure at a tissue site for a given negative-pressure source. In some example embodiments, the cover 140 may be a polymer drape, such as a polyurethane film, that is permeable to water vapor but impermeable to liquid. The cover 140 may have a high moisture-vapor transmission rate (MVTR) in some applications. For example, the MVTR may be at least 250 g/m\hat{ }2 per twenty-four hours in some embodiments (based on ASTM E96/E96M for upright cup measurement). Such drapes typically have a thickness in the range of 25-50 microns. For permeable materials, the permeability generally should be low enough that a desired negative pressure may be maintained. In some embodiments, the cover 140 may form an outer surface of the dressing 110.

An attachment device may be used to attach the cover 140 to an attachment surface, such as undamaged epidermis, a gasket, or another cover (e.g. at the tissue site). The attachment device may take many forms. For example, an attachment device may be a medically-acceptable, pressure-sensitive adhesive configured to bond the cover 140 to epidermis around a tissue site. In some embodiments, for example, some or all of the cover 140 may be coated with an adhesive, such as an acrylic adhesive, which may have a coating weight between 25-65 grams per square meter (g·s·m.). Thicker adhesives, or combinations of adhesives, may be applied in some embodiments to improve the seal and reduce leaks. Other example embodiments of an attachment device may include a double-sided tape, paste, hydrocolloid, hydrogel, silicone gel, or organogel.

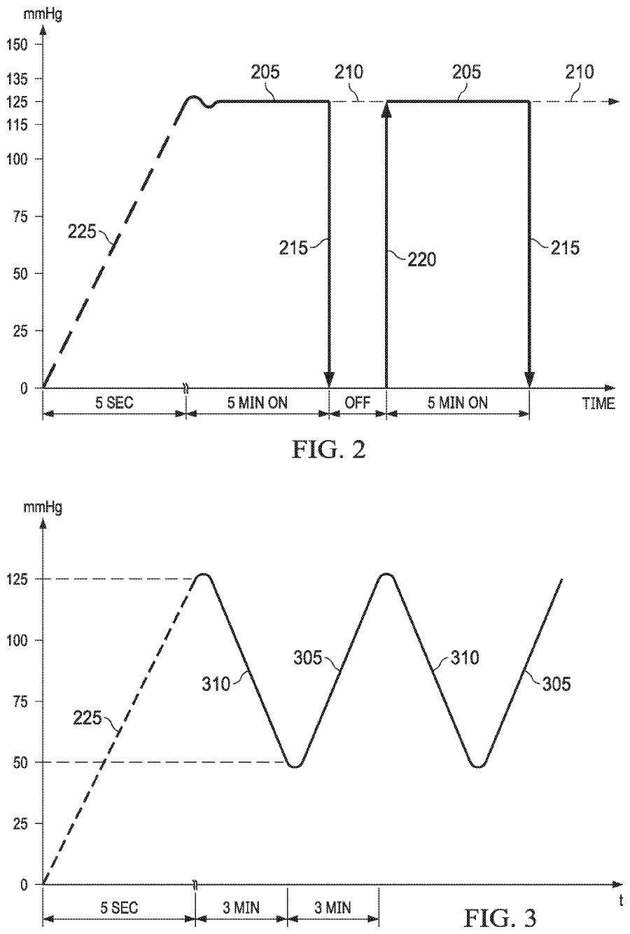

FIG. 2 is a graph illustrating additional details of an example control mode that may be associated with some embodiments of the controller 120. In some embodiments, the controller 120 may have a continuous pressure mode, in which the negative-pressure source 105 is operated to provide a constant target reduced pressure, as indicated by line 205 and line 210, for the duration of treatment or until manually deactivated. Additionally or alternatively, the controller may have an intermittent pressure mode, as illustrated in the example of FIG. 2. In FIG. 2, the x-axis represents time, and the y-axis represents reduced pressure generated by the negative-pressure source 105 over time. In the example of FIG. 2, the controller 120 can operate the negative-pressure source 105 to cycle between a target pressure and atmospheric pressure. For example, the target pressure may be set at a value of 125 mmHg, as indicated by line 205, for a specified period of time (e.g., 5 min), followed by a specified period of time (e.g., 2 min) of deactivation, as indicated by the gap between the solid lines 215 and 220. The cycle can be repeated by activating the negative-pressure source 105, as indicated by line 220, which can form a square wave pattern between the target pressure and atmospheric pressure.

In some example embodiments, the increase in negative-pressure from ambient pressure to the target pressure may not be instantaneous. For example, the negative-pressure source 105 and the dressing 110 may have an initial rise time, as indicated by the dashed line 225. The initial rise time may vary depending on the type of dressing and therapy equipment being used. For example, the initial rise time for one therapy system may be in a range of about 20-30 mmHg/second and in a range of about 5-10 mmHg/second for another therapy system. If the therapy system 100 is operating in an intermittent mode, the repeating rise time as indicated by the solid line 220 may be a value substantially equal to the initial rise time as indicated by the dashed line 225.

FIG. 3 is a graph illustrating additional details that may be associated with another example pressure control mode in some embodiments of the therapy system 100. In FIG. 3, the x-axis represents time and the y-axis represents negative pressure generated by the negative-pressure source 105. The target pressure in the example of FIG. 3 can vary with time in a dynamic pressure mode. For example, the target pressure may vary in the form of a triangular waveform, varying between a minimum and maximum reduced pressure of 50-125 mmHg with a rise time 305 set at a rate of +25 mmHg/min. and a descent time 310 set at −25 mmHg/min, respectively. In other embodiments of the therapy system 100, the triangular waveform may vary between reduced pressure of 25-125 mmHg with a rise time 305 set at a rate of +30 mmHg/min and a descent time 310 set at −30 mmHg/min.

In some embodiments, the controller 120 may control or determine a variable target pressure in a dynamic pressure mode, and the variable target pressure may vary between a maximum and minimum pressure value that may be set as an input prescribed by an operator as the range of desired reduced pressure. The variable target pressure may also be processed and controlled by the controller 120, which can vary the target pressure according to a predetermined waveform, such as a triangular waveform, a sine waveform, or a saw-tooth waveform. In some embodiments, the waveform may be set by an operator as the predetermined or time-varying reduced pressure desired for therapy.

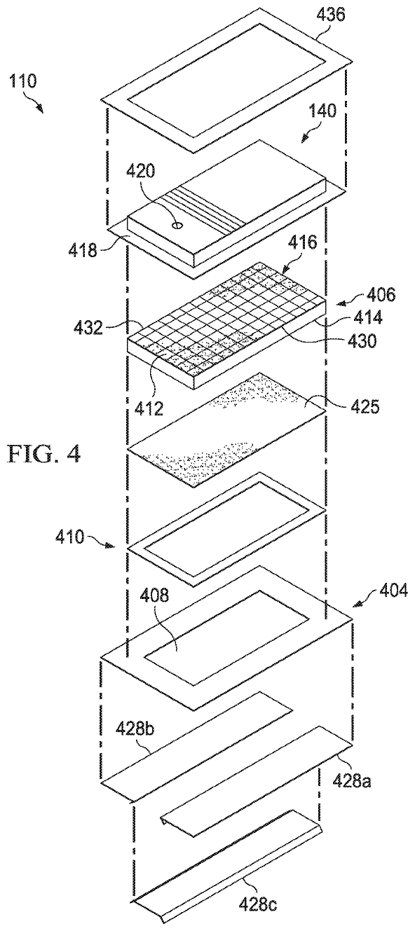

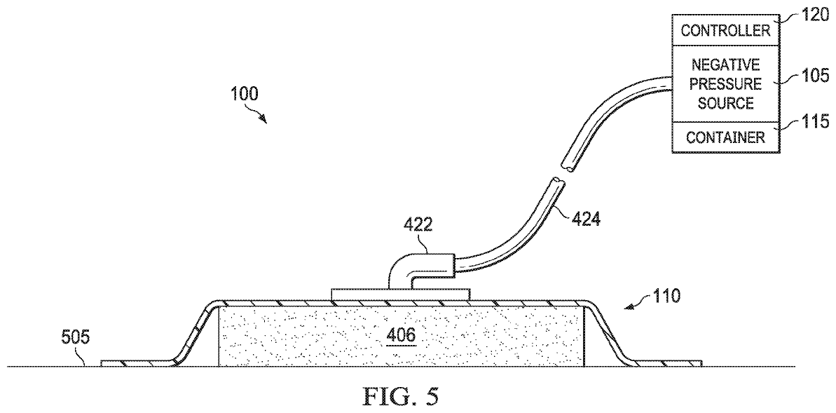

Referring to FIGS. 4-5, the dressing 110 may include features that can treat a tissue site, or parts thereof, and an area of tissue around the tissue/treatment site. For example, the tissue site may be an incision or other treatment target on a patient. The dressing 110 may be configured to treat not only the incision or treatment target, but also, an area of tissue around the incision or treatment target. While the figures may illustrate exemplary dressing embodiments with a particular longitudinal shape, other exemplary dressings may have other sizes, shapes, and/or configurations, for example for use on other tissue sites.

FIG. 4 is an exploded, isometric view of an example embodiment of a dressing that may be associated with an example embodiment of the therapy system of FIG. 1. Referring more specifically to FIG. 4, in some examples, the dressing 110 may include an attachment device 404, a manifold 406, and the cover 140. Some examples of the attachment device 404 and other components may include a treatment aperture 408, and the manifold 406 may be configured to be at least partially exposed to a tissue site through the treatment aperture 408. Further, in some examples, the dressing 110 may optionally include an adhesive ring 410 that may be configured to bond a peripheral portion of the manifold 406 to a portion of the attachment device 404. In some examples, the adhesive ring 410 may be formed as part of the attachment device 404, or the adhesive ring 410 may be omitted with the attachment device 404 instead being coupled to the manifold 406 and/or cover 140 with another medically acceptable coupling apparatus. In some examples, the cover 140, the manifold 406, the optional adhesive ring 410, and the attachment device 404 may have similar shapes. The attachment device 404 may be slightly larger than the manifold 406 to permit coupling of the attachment device 404 to the cover 140 around the manifold 406. In some examples, an adhesive may be disposed on a portion of the manifold 406 exposed through the treatment aperture 408. In some embodiments, the adhesive may be pattern-coated, and may cover up to 50% of the exposed portion or surface of the manifold 406.

The cover 140, the manifold 406, the attachment device 404, or various combinations may be assembled before application or at a tissue site. In some embodiments, the dressing 110 may be provided as a single unit.

The manifold 406 may include a first surface 414 and an opposing second surface 412. In some examples, at least a portion of the first surface 414 (e.g. the tissue-facing surface) of the manifold 406 may be configured to face the tissue site (e.g. the area of tissue around the extremity) through the treatment aperture 408. In some examples, the attachment device 404 may be positioned on or at a portion of the first surface 414 of the manifold 406. In some examples, the manifold 406 may include or be formed of a porous material, such as foam.

In some examples, the attachment device 404 may be configured to create a sealed space between the cover 140 and the tissue site, and the manifold 406 may be configured to be positioned in the sealed space. For example, the attachment device 404 may be positioned around an edge 416 of the manifold 406 and configured to surround the tissue site. The cover 140 may be disposed over the manifold 406 and coupled to the attachment device 404 around the manifold 406. For example, the cover 140 may be coupled to a portion of the attachment device 404 extending outward from the edge 416 of the manifold 406. Further, the cover 140 may be larger than the manifold 406, as illustrated in the example of FIG. 4, and may have a perimeter or a flange 418 configured to be attached to the attachment device 404. Assembled, the cover 140 may be disposed over the second surface 412 (e.g. the outward-facing surface) of the manifold 406, and the flange 418 may be attached to the attachment device 404 around the manifold 406. For example, an adhesive may be used to adhere the flange 418 to the attachment device 404, or the flange 418 may be, without limitation, welded, stitched, or stapled to the attachment device 404. In some embodiments, the attachment device may comprise an adhesive applied to the flange 418 and configured to allow attachment of the flange 418 to the tissue site. The cover 140 may also include a port 420 configured to allow fluid communication between the manifold 404 and a dressing interface 422 and/or a fluid conductor 424 (e.g. to apply negative pressure under the cover) as described herein.

The attachment device 404 may take many forms. In some examples, the attachment device 404 may include or be formed of a film or membrane that can provide a seal in a therapeutic negative-pressure environment. In some example embodiments, the attachment device 404 may be a polymer film, such as a polyurethane film, that is permeable to water vapor but impermeable to liquid. The attachment device 404 may have a thickness in the range of 25-50 microns. For permeable materials, the permeability may be low enough that a desired reduced pressure may be maintained. The attachment device 404 may also include a medically-acceptable adhesive, such as a pressure-sensitive adhesive. In examples, the attachment device 404 may be a polymer film coated with an adhesive, such as an acrylic adhesive, which may have a coating weight between 25-65 grams per square meter (g·s·m.). Thicker adhesives, or combinations of adhesives, may be applied in some examples to improve the seal and reduce leaks.

In some examples, the attachment device 404 may include or be formed of a hydrocolloid. In some examples, the attachment device 404 may be configured or referred to as a sealing ring or a gasket member. In other examples, the dressing 110 may include a gasket member (not shown) in addition to the attachment device 404. In such an example, the gasket member may be a peripheral member, such as a hydrocolloid ring, and at least a portion of the attachment device 404 may be positioned between the manifold 406 and the gasket member on or at a surface of the manifold 406, such as the first surface 414, configured to face the area of tissue around the tissue site. In some examples, the gasket member may have a similar or analogous shape as the adhesive ring 410, but the gasket member may be positioned on a surface of the attachment device 404 configured to face the tissue site such that the gasket member is configured to be positioned between the tissue site and the attachment device 404.

In some examples, the dressing 110 may optionally further include a protective layer 425, which may be coupled to a surface of the manifold 406, such as the first surface 414, and may be configured to be exposed to the tissue site. In some embodiments, the protective layer 425 may be configured to be positioned in direct contact with the tissue site, for example forming a tissue-contact surface. In other embodiments (e.g. without a protective layer), the tissue-contact surface may be formed by the manifold and/or the attachment device. The protective layer 425 may include or be formed of a material that substantially reduces or eliminates skin irritation while allowing fluid transfer through the protective layer. In some embodiments, the protective layer 425 may form a fluid control layer, configured to allow fluid communication between the tissue site and the manifold during negative-pressure therapy, while minimizing backflow of fluids (such as exudate) from the manifold to the tissue site (e.g. to minimize maceration). In some examples, the protective layer 425 may include or be formed of one or more of the following materials, without limitation: a woven material, a non-woven material, a polyester knit material, and a fenestrated film.

In some examples, the attachment device 404, which may comprise an adhesive on a surface of the dressing 110 configured to face the tissue site (e.g. on the tissue-contact surface), may be covered by one or more release liners 428 prior to applying the dressing 110 at the tissue site. For example, as shown in FIG. 4, the dressing 110 may include a first release liner 428a, a second release liner 428b, and a third release liner 428c. The first release liner 428a may be positioned proximate to a first side 430 of the manifold 406 or the dressing 110, the second release liner 428b may be positioned proximate to a second side 432 of the manifold 406 or the dressing 110 (e.g. with the first side 430 and the second side 432 opposite each other across a line of symmetry), and the third release liner 428c may be positioned proximate to a fold axis, centerline, or line or symmetry of the manifold 406 or the dressing 110 (e.g. spanning a central portion of the manifold and/or dressing). The central portion with the line of symmetry may be located between the first side 430 and the second side 432, and the third release liner 428c may be positioned between the first release liner 428a and the second release liner 428b. In some examples, the third release liner 428c may be configured to be removed to expose an adhesive or portion of the attachment device 404 proximate to the line of symmetry prior to removal of the first release liner 428a and the second release liner 428b. Such a configuration may permit the central portion of the dressing 110 (e.g. in proximity to the line of symmetry) to be initially positioned or aligned at a tissue site, such as the extremity, while the first release liner 428a and the second release liner 428b protect other portions of the adhesive or the attachment device 404. For example, a portion of the third release liner 428c may cover or be positioned over a portion of the first release liner 428a and/or the second release liner 428b such that the third release liner 428c may be removed prior to removal of the first release liner 428a and the second release liner 428b. In some examples, the dressing 110 may have two release liners, each of which may have perforations or slits (not shown here) configured to allow the release liners to be separated into smaller pieces for removal. Additionally, some embodiments may also have one or more casting sheet liners 436.

Additionally or alternatively, the first release liner 428a, the second release liner 428b, and the third release liner 428c may provide stiffness to the attachment device 404 to facilitate handling and application. Additionally or alternatively, the casting sheet liners 436 may cover the flange 418 to provide stiffness to the cover 140 for handling and application. The one or more release liner 428 may be configured to releasably cover the attachment device 404, for example to protect and maintain the adhesive of the attachment device 404 until the time of application of the dressing 110 to the tissue site.

FIG. 5 is a schematic view illustrating an exemplary system 100 including a simplified example of the dressing 110 in place on an exemplary tissue site 505, illustrating additional details that may be associated with some embodiments. The system 100 may comprise a negative-pressure source 105 in fluid communication with the dressing 110. For example, the dressing 110 may comprise a dressing interface 422, which may penetrate or fluidly couple with the dressing 110 through the port 420 in the cover 140 to fluidly couple to the manifold 406 of the dressing 110. In some embodiments, a fluid conductor 424 may fluidly couple the negative-pressure source 105 to the dressing interface 422 (thereby fluidly coupling the negative-pressure source 105 to the manifold 406 of the dressing 110, for application of negative-pressure therapy to the tissue site 505 through the manifold 406 of the dressing 110).

In operation, the negative-pressure source 105 can reduce pressure in the sealed therapeutic environment (e.g. when the dressing 110 is applied to the tissue site 505 in the usage configuration). Reduced pressure applied to the tissue site 505 through the manifold 406 in the sealed therapeutic environment can induce macro-strain and/or micro-strain in the tissue site, as well as remove exudates and other fluids from the tissue site 505, which can be collected in the container 115.

In general, exudates and other fluids flow toward lower pressure along a fluid path. Thus, the term “downstream” may refer to a location in a fluid path relatively closer to a source of reduced pressure or further away from a source of positive pressure. Conversely, the term “upstream” may refer to a location further away from a source of reduced pressure or closer to a source of positive pressure.

In some example embodiments, the controller 120 may receive and process data from one or more sensors, such as the first sensor 125. The controller 120 may also control the operation of one or more components of the therapy system 100 to manage the pressure delivered to the tissue interface 135, such as the manifold 406 and associated components. In some embodiments, the controller 120 may include an input for receiving a desired target pressure, and may be programmed for processing data relating to the setting and inputting of the target pressure to be applied to the tissue interface 135. In some example embodiments, the target pressure may be a fixed pressure value set by an operator as the target reduced pressure desired for therapy at a tissue site and then provided as input to the controller 120. The target pressure may vary from tissue site to tissue site based on the type of tissue forming a tissue site, the type of injury or wound (if any), the medical condition of the patient, and the preference of the attending physician. After selecting a desired target pressure, the controller 120 can operate the negative-pressure source 105 in one or more control modes based on the target pressure, and may receive feedback from one or more sensors to maintain the target pressure at the tissue interface 135. In some embodiments, the manifold 406 may have distinct pressure zones, and different target pressures and control modes may be applied to different pressure zones.

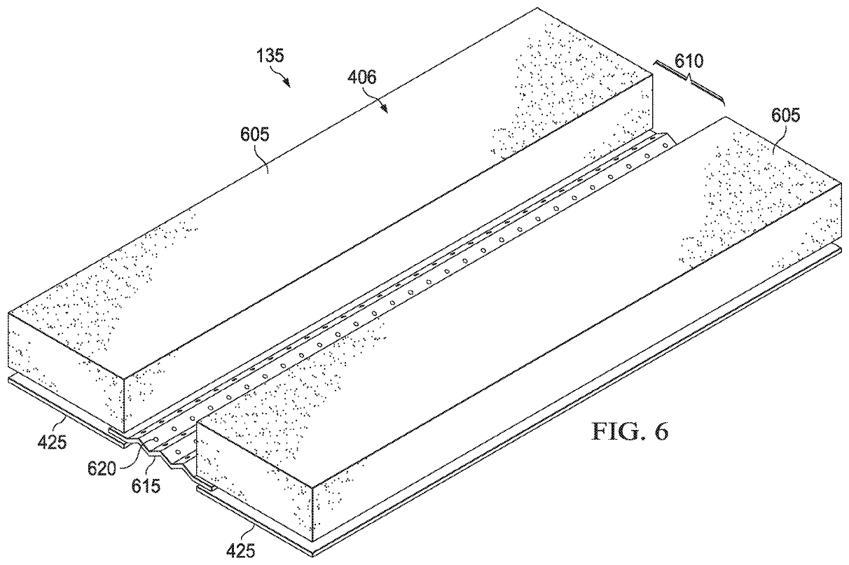

FIG. 6 is an isometric view of an example tissue interface 135 for a dressing (which may be similar to that of FIG. 4) for use with the therapy system of FIG. 1, illustrating additional details that may be associated with some embodiments. In the embodiment of FIG. 6, the manifold 406 may comprise two strips 605 of manifolding material, which may be horizontally-spaced with a gap 610 therebetween. For example, the two strips 605 of manifolding material may be located laterally side-by-side, with the gap therebetween. In some embodiments, the two strips 605 would not be vertically stacked, but for example may be laterally offset and spaced apart with no overlap. In some embodiments, the first surface 414 of each of the two strips 605 may be configured to be parallel to each other and offset laterally across the gap 610, for example with the first surface 414 of the two strips 605 configured to be approximately parallel to the tissue site and the gap 610 between the two strips configured to provide a view of the tissue site unobstructed by the manifold. In some embodiments, a separator film 615 may span the gap 610 and be bonded to the two strips 605 of manifolding material, thereby defining the gap 610 between the two strips 605 of manifolding material. In some embodiments, the separator film 615 may be substantially transparent, forming a viewing window between the two strips 605 of manifolding material. In some embodiments, the viewing window may be configured to allow an underlying incision on the tissue site to be seen, for example substantially in its entirety. In some embodiments, the manifolding material of the two strips 605 may be configured to manifold or distribute negative pressure, to draw down (e.g. compress) under negative pressure, and/or to provide lateral strain and/or appositional forces at the tissue site under negative pressure. For example, the manifolding material may comprise open-cell foam. In some embodiments, the tissue interface 135 as a whole may be configured to provide lateral strain and/or appositional forces to substantially the entirety of the tissue site during negative-pressure therapy, for example drawing an incision on the tissue site and under the viewing window (e.g. within the gap 610 and/or under the separator layer 615) towards a closed position.

In some embodiments, the gap 610 may have a width of about 8-12 mm. In some embodiments, the separator film 615 may comprise polyurethane (PU), polyethylene (PE), or silicone. In some embodiments, the separator film 615 may have a thickness of about 50-150 micron. Some embodiments of the separator film 615 may comprise a plurality of perforations 620.

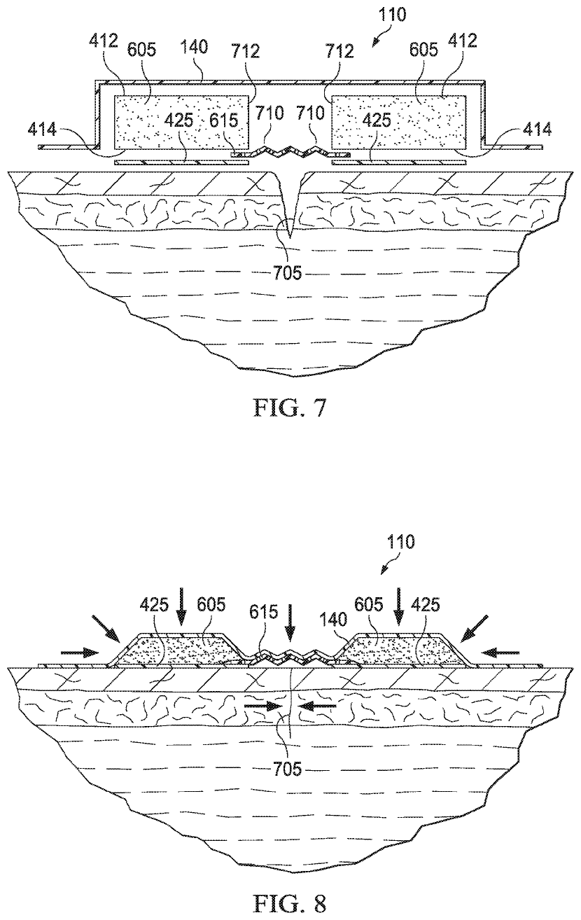

FIG. 7 is a schematic cross-section view of an example dressing 110 having the tissue interface 135 of FIG. 6, illustrating additional details that may be associated with some embodiments. In some embodiments, the gap 610 may be defined between the two strips 605 of manifolding material, for example extending laterally between the interior sidewalls 712 of the strips 605 (e.g. with the interior sidewalls 712 of the two strips 605 approximately parallel to each other and/or facing each other across the gap 610). In some embodiments, the interior sidewalls 712 may extend approximately perpendicularly between the first surface 414 and the second surface 412 of the strips 605. In some embodiments, the separator film 615 may comprise the plurality of perforations 620, which may substantially span its surface. For example, the plurality of perforations 620 may be substantially co-extensive with the entirety of the separator film 615. In some embodiments, each of the perforations 620 may be a hole with a diameter of about 0.5-1 mm. In some embodiments, the perforations 620 may be spaced apart about 2-5 mm, for example with about 2-3 mm between adjacent perforations 620. In some embodiments, each of the perforations may be a slit, for example having a length of about 2-3 mm.

In some embodiments, the separator film 615 may be configured with and/or may comprise textured features. For example, the textured features may comprise one or more longitudinal ridges 710 with one or more peaks. In some embodiments, the ridges 710 may be thermoformed. In some embodiments, the ridges 710 may be configured to collapse and/or to provide lateral strain and/or appositional forces to an area of the tissue site (e.g. an incision 705) located under the viewing window (e.g. the gap 610 and/or the separator film 615) during negative-pressure therapy. In some embodiments, the ridges 710 may extend substantially parallel to the longitudinal centerline and/or the strips 605 of manifolding material. In some embodiments, one or more of the ridges 710 may jut into the gap 610, for example extending upward between the two strips 605 of manifolding material. In some embodiments, the ridges 710 may be spaced about 1-2 mm between peaks. In some embodiments, the perforation slits of the separator film 615 may extend substantially parallel to the ridges 710 and/or to a longitudinal centerline of the gap 610.

In some embodiments, the separator film 615 may be bonded to a first (tissue/inward-facing) surface 414 of the two strips 605 of manifolding material. In some embodiments, the separator film 615 may be approximately parallel to the first surface 414 of the two strips 605 and/or may overlap with each of the two strips 605 of manifolding material by about 3-5 mm, for example on an underside (e.g. the first surface 414) of the two strips 605 of manifolding material. In some embodiments, the portions of the separator layer 615 which overlap with the two strips 605 may be untextured, for example substantially flat.

Some embodiments may further comprise a protective layer 425 adjacent to the first surface 414 of the two strips 605 of manifolding material. In some embodiments, the protective layer 425 may form at least part of the tissue-contact surface for the dressing 110. In some embodiments, the protective layer 425 may be configured to prevent tissue in-growth into the strips 605 of manifolding material, while allowing communication of negative pressure between the strips 605 of manifolding material and the tissue site. In some embodiments, the protective layer 425 may not be textured. In some embodiments, the protective layer 425 may be bonded to the two strips 605 of manifolding material and/or to the separator film 615. In some embodiments, the separator film 615 may be bonded between the strips 605 of manifolding material and the protective layer 425. In some embodiments, the protective layer 425 and the separator layer 615 may be jointly formed by a unitary film, wherein the portion of the unitary film spanning the gap 610 may be thicker (e.g. about 50-150 micron) than the portions of the unitary film spanning the two strips 605 of manifolding material (which may be about 20-50 micron). For example, the unitary film may form both the separator film 615 and the protective layer 425.

Some embodiments may further comprise a cover 140 configured to be disposed over the two strips 605 of manifolding material and to substantially prevent fluid flow through the cover 140 material. When attached to the tissue site, the cover 140 may be configured to seal for negative-pressure therapy. In some embodiments, the cover 140 may be disposed adjacent to the second (e.g. outward-facing) surface 412 of the two strips 605 of manifolding material. In some embodiments, the cover 140 may be substantially transparent. In some embodiments, only open space may be located vertically between the cover 140 and the separator film 615, for example in the absence of negative pressure. For example, no support structures may extend between the cover 140 and the separator film 615, within the gap. In some embodiments, the transparent cover 140 and the transparent separator film 615 may jointly form the viewing window through the gap 610 between the two strips 605 of manifolding material. In some embodiments, the cover 140 may comprise polyurethane (PU) film.

Optionally, some dressing embodiments may further comprise an absorbent layer (not shown here). For example, the absorbent layer may be located one either or both sides of the gap 610, and/or may be located between at least one of the two strips 605 of manifolding material and the cover 140. In some embodiments, the absorbent layer may comprise absorbent material, such as SAP, between layers of wicking material, forming a pouch. In some embodiments, the absorbent material may be located between the two strips 605 of manifolding material and cover 140, with adhesive bonding the cover 140 to the separator film 615 or the two strips 605 of manifolding material to form pouches which may contain the absorbent material.

In some embodiments, the separator film 615 and/or the protective layer 425 may optionally be coated with one or more of the following, for example on the first (e.g. inward, tissue-facing) surface 414: oxysalt, citric acid, silver, and an anti-microbial agent. Optionally, some embodiments may further comprise a sensor configured to indicate the presence of bacteria, for example of high Protease activity. Some embodiments optionally may further comprise one or more hydration sensors, which may be configured to indicate skin hydration level. For example, the one or more hydration sensors may be placed on one or more end of the gap and/or along the viewing window and/or over the strips 605 of manifolding material.

FIG. 8 is a schematic cross-section view of the dressing of FIG. 7 during negative-pressure therapy on a tissue site, illustrating additional details that may be associated with some embodiments. In some embodiments, the cover 140 may be configured to collapse to contact the separator film 615 in the gap 610 under negative pressure (e.g. during negative-pressure therapy using the dressing 110). FIG. 8 illustrates this collapse during negative-pressure therapy, during which negative pressure in the dressing 110 draws down the cover 140 into contact with the separator film 615. In some embodiments, the collapse of the cover 140 into contact with the separator film 615 may form creases and/or manifolding channels in proximity to the separator film 615. In other embodiments, the cover 140 may be coupled (e.g. adhered) to the separator film 615, such that FIG. 8 may illustrate the dressing regardless of the negative pressure within the dressing 110.

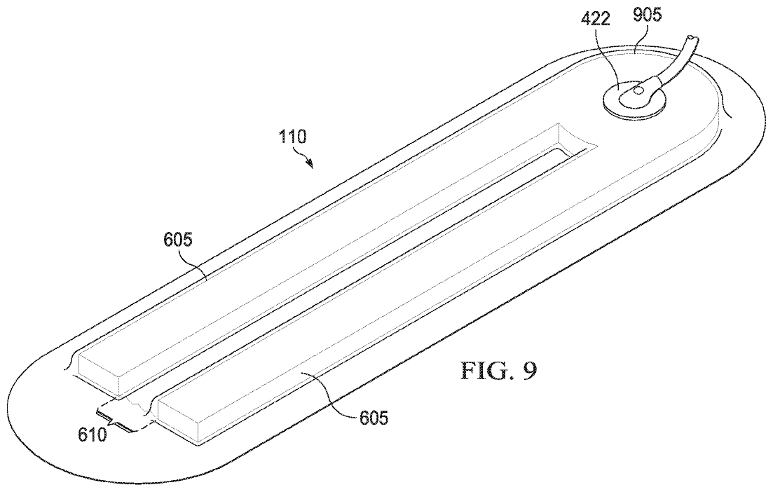

FIG. 9 is an isometric view of another example dressing similar to that of FIG. 7 for use with the therapy system of FIG. 1, illustrating additional details that may be associated with some embodiments. In some embodiments, the two strips 605 of manifolding material may be joined at one or both ends, forming a joined area 905. For example, FIG. 9 illustrates the two strips 605 joined at a single end by joined area 905. In some embodiments, the joined area 905 may comprise one or more of the following: a manifolding material (for example, configured to provide fluid communication between the two strips 605), a gel, or an area of encapsulated air. In some embodiments, the two strips 605 of manifolding material and the joined area 905 of manifolding material may form a unitary element, with the gap 610 extending only part of the length of the dressing 110 between the two strips 605 but not extending into the joined area 905. For example, the gap 610 may not extend the full length of the dressing 110, but may extend from a first end of the strips 605 (away from the joined end 905) to the joined area 905. In some embodiments, the joined area 905 may span the gap 610, for example at one or more ends of the dressing 110. Some embodiments may further comprise a dressing interface 422, which may be fluidly coupled to the joined area 905. For example, the dressing interface 422 may be configured to fluidly couple the two strips 605 of manifolding material to a negative-pressure source, for instance through the cover 140 and/or the joined area 905. In some embodiments, the joined area 905 may be configured to offload the forces from the dressing interface 422, for example to increase patient comfort.

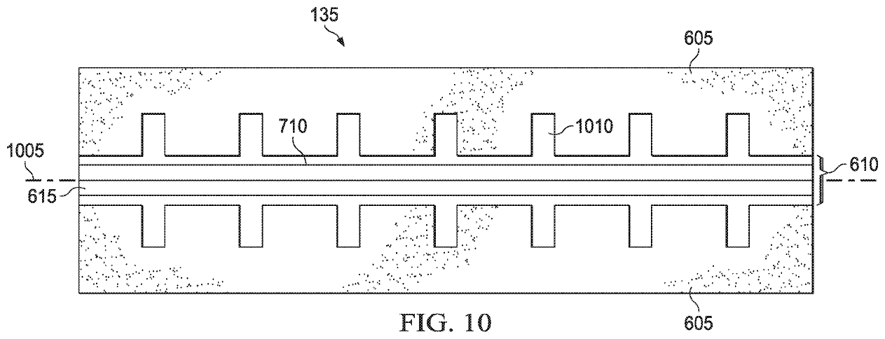

FIG. 10 is a bottom plan view of another example tissue interface 135, illustrating additional details that may be associated with some embodiments. As FIG. 10 illustrates, the gap 610 may extend longitudinally between the two strips 605 of manifolding material, with a longitudinal centerline 1005. In some embodiments, the longitudinal centerline 1005 of the gap 610 may correspond to the longitudinal centerline of the dressing 110. In some embodiments, the viewing window may further comprise horizontal strips of open and/or viewing space. For example, the viewing window may comprise one or more horizontal recess openings 1010. The horizontal recess openings 1010 may extend outward from the longitudinal opening of the gap 610 into one or both of the strips 605 of manifolding material and may penetrate and/or extend vertically through the strips 605 of manifolding material. In some embodiments, the horizontal recess openings 1010 may extend laterally approximately perpendicularly from the longitudinal centerline of the gap 610. In some embodiments, pairs of horizontal recess openings 1010 may be located opposite each other across the longitudinal centerline 1005. In some embodiments, at least two of the horizontal recess openings 1005 may be spaced longitudinally, forming two or more horizontal strips of viewing space of the viewing window. In some embodiments, the width of the horizontal recess openings 1005 may be less than the width of the gap 610. In some embodiments, the separator film 615 may span both the gap 610 and the horizontal recess openings 1010. In some embodiments, the textured features of the separator film 615, such as ridges 710, may only be located within the gap 610, and may not extend into the horizontal recess openings 1010. The tissue interface 135 of FIG. 10 may be used within a dressing similar to that of FIG. 7, in some embodiments.

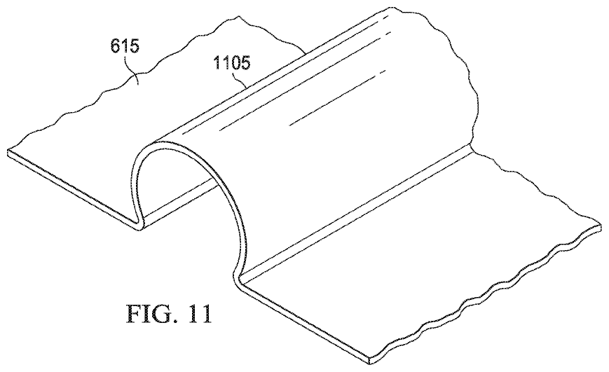

FIG. 11 is a sectional isometric view of an example separator film 615 that may be associated with some embodiments of the tissue interface of FIG. 6, illustrating additional details that may be associated with some embodiments. In some embodiments, the textured features may comprise a conduit 1105. In some embodiments, the conduit 1105 may be configured to draw in at a lower surface level (e.g. in proximity to the tissue site) when negative-pressure therapy is applied to the dressing, imparting lateral and/or appositional forces to the incision of the tissue site due to the channel shape and/or configuration. For example, the conduit 1105 may have a diameter of about 2-3 mm and/or a film thickness of about twice the thickness of the remainder of the separator film 615. In some embodiments, the conduit 1105 may be shaped as about ⅔ of a longitudinally extending polymeric film tube, for example without the bottom ⅓ of the tube. The open bottom (e.g. forming the lower surface level) of the tube may be attached to the remainder of the separator film 615, for example forming a conduit opening which may be configured to face towards the tissue site during negative-pressure therapy. In some embodiments, the conduit 1105 may be in addition to the textured features, such as thermoformed ridges. In some embodiments, the conduit 1105 may be formed as a separate tube, the bottom portion (for example, the bottom ⅓) of the tube may be removed (for example, by cutting), and then the bottom of the tube may be coupled to a conduit gap in the separator film 615. In some embodiments, the conduit 1105 may be thermoformed, for example as part of the separator film 615. When evacuated during negative-pressure therapy, the conduit 1105 may be configured to collapse and/or flex inward at the lower surface level, for example providing lateral strain and/or appositional forces to the tissue site. In some embodiments, the conduit 1105 may be configured so that, when negative-pressure therapy is applied to the dressing, the conduit 1105 may collapse inward laterally so that the conduit gap may be reduced by about half of its width (for example with the bottom of the conduit 1105 flexing inward). In some embodiments, the conduit 1105 may extend longitudinally in proximity to the longitudinal centerline of the gap or in proximity to a side of the gap.

In use, method embodiments for providing negative-pressure therapy to a tissue site may comprise: positioning a dressing with a viewing window over the tissue site; and applying negative pressure through the dressing to the tissue site. In some embodiments, the viewing window may be positioned over an incision, for example substantially over the entire length of the incision. In some embodiments, the negative pressure may collapse the dressing vertically and laterally in the horizontal direction. Some embodiments may further comprise using (e.g. viewing through the window) the viewing window to position the dressing over the incision. Some embodiments may further comprise viewing the tissue site through the view window during application of negative pressure, with the gap between the strips of manifolding material being held open by a transparent separator film. In some embodiments, applying negative pressure may induces lateral strain and/or appositional forces to the tissue site, for example closing or drawing closed the incision. For example, the configuration of the separator film may allow lateral strain and/or appositional forces to be transmitted to the incision under the viewing window, even while the viewing window is held open. In some embodiments, applying negative pressure to the dressing may remove fluids, such as exudate, from the tissue site. In some embodiments, applying negative pressure may collapse the cover of the dressing into proximity with the separator film (e.g. the cover may be drawn down into contact with the separator film) to provide an optically clear viewing window.

Also disclosed are examples of methods of manufacturing a negative-pressure dressing, similar to the embodiments described herein, which may comprise: providing two manifolding strips; disposing the two manifolding strips laterally side-by-side with a gap therebetween; providing a separator film; disposing the separator film to span the gap; and bonding the separator film to each of the manifolding strips. In some embodiments, providing the separator film may comprise: providing a substantially transparent film; forming perforations in the film; and forming textured features in the film. In some embodiments, forming textured features may comprise thermoforming the film to form longitudinal ridges. In some embodiments, the separator film may be bonded to a first surface of the strips; and the method may comprise attaching a protective layer to the first surface of the strips. In some embodiments, the separator film may extend to span the width of both manifolding strips and the gap (e.g. as a unitary film which may serve as both the separator film and the protective layer). For example, the separator film may be thicker in a central portion (e.g. spanning the gap) and thinner at perimeter portions (e.g. portions spanning the manifolding strips). In some embodiments, providing the separator film may further comprise forming the substantially transparent film to be thicker at the central portion and thinner at the portions spanning the strips. In some embodiments, the portions of the separator film spanning the strips may not have textured features. For example, forming the textured features may comprise forming textured features only on the portion of the separator film spanning the gap. Some embodiments may further comprise providing a cover and disposing the cover over the manifolding strips (e.g. on the second surface). In some embodiments, providing two manifolding strips may comprise forming two manifolding strips having horizontal recess openings on a side configured to face the gap. Some embodiments may further comprise providing a conduit with a film thickness about twice that of the separator film and coupling the conduit to the separator film. In some embodiments, providing a conduit may comprise providing a conduit film and thermoforming the conduit film into an open conduit, for example a longitudinal tube with a diameter of about 2-3 mm and/or an open bottom ⅓.

The systems, apparatuses, and methods described herein may provide significant advantages. For example, in addition to the benefits of increased development of granulation tissue and reduced healing times, some system 100 embodiments may allow observation of the incisional wound site during therapy. In some embodiments, the configuration may allow the user to view associated near peri-wound areas as well. In some embodiments, the configuration may allow for manifolding of pressure and/or fluids through the dressing during negative-pressure therapy. In some embodiments, the configuration may allow the incision to experience apposition forces and/or lateral strain, of the sort which may close the incision during negative-pressure therapy, despite the viewing window over the incision and/or while allowing the user to view the incision. In some embodiments, the viewing window may allow the user to view substantially the entire incision and/or may allow unobstructed visualization of the wound.

If something is described as “exemplary” or an “example”, it should be understood that refers to a non-exclusive example. The terms “about” or “approximately” or the like, when used with a number, may mean that specific number, or alternatively, a range in proximity to the specific number as understood by persons of skill in the art field (for example, +/−10%). Use of broader terms such as “comprises”, “includes”, and “having” should be understood to provide support for narrower terms such as “consisting of”, “consisting essentially of”, and “comprised substantially of”. Use of the term “optionally”, “may”, “might”, “possibly”, “could”, “can”, “would”, “should”, “preferably”, “typically”, “often” and the like with respect to any element, component, feature, characteristic, etc. of an embodiment means that the element, component, feature, characteristic, etc. is not required, or alternatively, the element, component, feature, characteristic, etc. is required, both alternatives being within the scope of the embodiment(s). Such element, component, feature, characteristic, etc. may be optionally included in some embodiments, or it may be excluded (e.g. forming alternative embodiments, all of which are included within the scope of disclosure). Section headings used herein are provided for consistency and convenience, and shall not limit or characterize any invention(s) set out in any claims that may issue from this disclosure. If a reference numeral is used to reference a specific example of a more general term, then that reference numeral may also be used to refer to the general term (or vice versa).

While shown in a few illustrative embodiments, a person having ordinary skill in the art will recognize that the systems, apparatuses, and methods described herein are susceptible to various changes and modifications that fall within the scope of the appended claims. Moreover, descriptions of various alternatives using terms such as “or” do not require mutual exclusivity unless clearly required by the context, and the indefinite articles “a” or “an” do not limit the subject to a single instance unless clearly required by the context. Components may be also be combined or eliminated in various configurations for purposes of sale, manufacture, assembly, or use. For example, in some configurations the dressing, the container, or both may be eliminated or separated from other components for manufacture or sale. In other example configurations, the controller may also be manufactured, configured, assembled, or sold independently of other components.

The appended claims set forth novel and inventive aspects of the subject matter described above, but the claims may also encompass additional subject matter not specifically recited in detail. For example, certain features, elements, or aspects may be omitted from the claims if not necessary to distinguish the novel and inventive features from what is already known to a person having ordinary skill in the art. Features, elements, and aspects described in the context of some embodiments may also be omitted, combined, or replaced by alternative features serving the same, equivalent, or similar purpose without departing from the scope of the invention defined by the appended claims. Also, features, elements, and aspects described with respect to a particular embodiment may be combined with features, elements, and aspects described with respect to one or more other embodiments.

1. A dressing for applying negative-pressure therapy at a tissue site, comprising:
a manifold comprising two horizontally-spaced strips of manifolding material;
a gap between the two strips of manifolding material; and
a separator film spanning the gap and bonded to the two strips of manifolding material;
wherein the separator film is substantially transparent.

2. (canceled)

3. The dressing of claim 1, wherein the separator film comprises PU, PE or silicone.

4. (canceled)

5. The dressing of claim 1, wherein the separator film comprises a plurality of perforations substantially spanning the separator film.

6. The dressing of claim 5, wherein each of the plurality of perforations is a hole with diameter of about 0.5-1 mm; and the plurality of perforations are spaced apart about 2-5 mm.

7. The dressing of claim 5, wherein each of the plurality of perforations is a slit having a length of about 2-3 mm; and each slit is oriented to extend with the length substantially parallel to the two strips of manifolding material.

8. The dressing of claim 5, wherein the separator film is configured with textured features.

9. The dressing of claim 8, wherein the textured features comprise one or more thermoformed longitudinal ridges.

10. (canceled)

11. The dressing of claim 8, wherein the separator film is bonded to a first surface of the two strips of manifolding material.

12. (canceled)

13. The dressing of claim 11, further comprising a protective layer adjacent to the first surface of the two strips of manifolding material; wherein the protective layer is configured to prevent tissue in-growth into the strips of manifolding material while allowing fluid communication between the strips of manifolding material and the tissue site.

14. The dressing of claim 1, wherein the two strips of manifolding material are joined at one or both ends, and the gap does not extend the full length of the two strips of manifolding material.

15. The dressing of claim 1, further comprising a cover configured to be disposed over the two strips of manifolding material and to substantially prevent fluid flow therethrough; wherein the cover is substantially transparent.

16. The dressing of claim 15, wherein the cover is disposed adjacent to a second surface of the two strips of manifolding material.

17. A method for providing negative-pressure therapy to an incision, comprising:
positioning a dressing with a viewing window over the incision; and
applying negative pressure through the dressing to the incision, wherein the negative pressure collapses the dressing vertically and laterally in the horizontal direction and induces lateral strain and/or appositional forces on the incision.

18. (canceled)

19. A method of manufacturing a negative-pressure dressing, comprising:
providing two manifolding strips;
disposing the two manifolding strips laterally side-by-side with a gap therebetween;
providing a separator film;
disposing the separator film to span the gap; and
bonding the separator film to each of the manifolding strips.

20. The method of claim 19, wherein providing the separator film comprises:
providing a substantially transparent film;
forming perforations in the film; and
forming textured features in the film configured to provide lateral strain and/or appositional forces when under negative pressure.

21. The dressing of claim 1, wherein the separator film spanning the gap forms a viewing window between the two strips of manifolding material.

22.-24. (canceled)

25. The dressing of claim 13, wherein the protective layer and the separator film are jointly formed by a unitary film, wherein a central portion of the unitary film spanning the gap is thicker than lateral portions of the unitary film spanning the two strips of manifolding material.

26. The dressing of claim 25, wherein the central portion of the unitary film has a thickness of about 50-150 micron, and the lateral portions of the unitary film have a thickness of about 20-5 micron.

27.-30. (canceled)

31. The dressing of claim 15, wherein the cover is configured to collapse to contact the separator film in the gap under negative pressure and to form creases and manifolding channels in proximity to the separator film.

32. (canceled)

33. The dressing of claim 8, wherein the textured features comprise a thermoformed conduit, having a diameter of about 2-3 mm and a film thickness of about twice the thickness of the remainder of the separator film.

34. The dressing of claim 33, wherein the thermoformed conduit extends longitudinally in proximity to a longitudinal centerline of the gap or in proximity to a side of the gap.

35. The dressing of claim 8, further comprising a thermoformed conduit formed of a polymeric film, having a diameter of about 2-3 mm and a film thickness of about twice the thickness of the separator film; wherein the thermoformed conduit is coupled to the separator film.

36.-38. (canceled)

39. The dressing of claim 21, further comprising one or more horizontal recess openings extending outward from the gap into one or both of the strips of manifolding material; wherein each of the horizontal recess openings extends vertically through the one or both strips to form horizontal sections of the viewing window.

40. The dressing of claim 39, wherein each of the horizontal recess openings extends approximately perpendicularly from a longitudinal centerline of the gap.

41.-44. (canceled)

45. A system for providing negative-pressure therapy to a tissue site, comprising:
a negative-pressure source; and
a dressing comprising:
a manifold comprising two horizontally-spaced strips of manifolding material;
a gap between the two strips of manifolding material;
a separator film spanning the gap and bonded to the two strips of manifolding material; and
a cover configured to be disposed over the manifold and to substantially prevent fluid flow therethrough
wherein:
the separator film and the cover are each substantially transparent; and
the negative-pressure source is fluidly coupled to the manifold through the cover.

46. The system of claim 45, wherein the separator film comprises a plurality of perforations substantially coextensive with the separator film.

47. The system of claim 45, wherein the separator film is configured with textured features.

48. The system of claim 47, wherein the textured features comprise one or more thermoformed longitudinal ridges.

49. The system of claim 45, wherein the dressing further comprises a protective layer adjacent to a first surface of the two strips of manifolding material.

50.-68. (canceled)
